(19) United States
(12) Patent Application Publication
(10) Pub. No.: US 2023/0210441 A1
(43) Pub. Date: Jul. 6, 2023

(54) BRAIN IMAGE ANALYSIS APPARATUS, CONTROL METHOD, AND COMPUTER READABLE MEDIUM

(71) Applicants: NEC CORPORATION, Tokyo (JP); OSAKA UNIVERSITY, Suita-shi, Osaka (JP)

(72) Inventors: Masataka KIKUCHI, Osaka (JP); Yasusi MATSUMURA, Osaka (JP); Kaori KOBAYASHI, Tokyo (JP); Kenichi KAMIJO, Tokyo (JP); Yasuto FUSHIMI, Tokyo (JP)

(73) Assignees: NEC CORPORATION, Tokyo (JP); OSAKA UNIVERSITY, Suita-shi, Osaka (JP)

(21) Appl. No.: 17/928,855

(22) PCT Filed: Jun. 1, 2020
(86) PCT No.: PCT/JP2020/021575

§ 371 Date: Nov. 30, 2022

(51) Int. Cl. A61B 5/00

(52) U.S. Cl. CPC A61B 5/4088; A61B 5/4064; A61B 5/743

(57) ABSTRACT

A brain image analysis apparatus (2000) acquires input data (40) including a structural brain image (42) and a functional brain image (44) for a subject (10). The brain image analysis apparatus (2000) obtains analysis data (20) by inputting the input data (40) into an analysis model (2020). The analysis model (2020) has been trained in advance so as to output the analysis data (20) representing information about brain dysfunction in response to an input of the input data (40). The brain image analysis apparatus (2000) outputs, based on the analysis data (20), output data (30) representing information about the brain dysfunction of the subject (10).

### TECHNICAL FIELD

The present invention relates to an analysis of a brain image.

### BACKGROUND ART

Technologies for finding out brain conditions by using brain images have been developed. For example, PTL1 discloses a technology for analyzing an MRI image of a patient suffering from mild cognitive disorder to predict the onset of dementia. Further, PTL2 discloses a technology for imaging the receptor binding capacity of a brain by using an image showing the structure of the brain (such as an MRI image) and an image of the brain captured by using a certain radioactive chemical agent that selectively binds to a receptor (such as a PET image). In the system disclosed in PTL2, after a region of interest in the PET image is determined by using the MRI image, a distribution of the receptor binding capacity of the brain is calculated by analyzing the PET image.

### CITATION LIST

### Patent Literature

PTL1: International Patent Publication No. WO2019/208661

PTL2: Japanese Unexamined Patent Application Publication No. 2013-061196

### SUMMARY OF INVENTION

### Technical Problem

In the invention disclosed in PTL1, dementia is predicted by using only information obtained from one type of image. In this regard, the inventors of the present application have found that information that can be used for the analysis of brain dysfunction is not limited to information obtained from one type of image. Note that in PTL2, an analysis is made by using two types of brain images. However, a technology disclosed by PTL2 is not one for performing an analysis regarding brain dysfunction, but one for determining a region of interest in a PET image by using an MRI image.

The present invention has been made in view of the above-described problem, and an object thereof is to provide a technology for improving the accuracy of an analysis of brain dysfunction using brain images.

### Solution to Problem

A brain image analysis apparatus according to the present disclosure includes: an analysis model that has been trained to output analysis data representing information about brain dysfunction in response to an input of input data including a structural brain image showing a structure of a brain and a functional brain image showing a functional state of the brain; an acquisition unit configured to acquire the input data for a subject; and an output processing unit configured to acquire the analysis data about the subject from the analysis model by inputting the acquired input data into the analysis model, and to output output data representing information about brain dysfunction of the subject based on the acquired analysis data.

A control method according to the present disclosure is performed by a computer. The computer includes an analysis model that has been trained to output analysis data representing information about brain dysfunction in response to an input of input data including a structural brain image showing a structure of a brain and a functional brain image showing a functional state of the brain.

The control method includes: an acquisition step for acquiring the input data for a subject; and an output processing step for acquiring the analysis data about the subject from the analysis model by inputting the acquired input data into the analysis model, and outputting output data representing information about brain dysfunction of the subject based on the acquired analysis data.

A computer readable medium according to the present invention stores a program for causing a management apparatus to perform a control method according to the present invention.

### Advantageous Effects of Invention

According to the present invention, a technology for improving the accuracy of an analysis of brain dysfunction using brain images is provided.

### BRIEF DESCRIPTION OF DRAWINGS

FIG. 1 shows an example of an overview of operations performed by a brain image analysis apparatus according to a first example embodiment;

FIG. 2 is a block diagram showing an example of a functional configuration of the brain image analysis apparatus according to the first example embodiment;

FIG. 3 is a block diagram showing an example of a hardware configuration of a computer that implements a brain image analysis apparatus;

FIG. 4 is a flowchart showing an example of a flow of processes performed by the brain image analysis apparatus according to the first example embodiment;

FIG. 5 shows an example of an analysis model generated using heterogeneous mixture learning;

FIG. 6 shows an example of output data;

FIG. 7 shows an example of a comparison of a result of an analysis made on a subject and with an existing case;

FIG. 8 is a first diagram showing an example of information obtained through training of an analysis model; and

FIG. 9 is a second diagram showing an example of information obtained through training of an analysis model.

### EXAMPLE EMBODIMENT

An example embodiment according to a present disclosure will be described hereinafter in detail with reference to the drawings. The same reference numerals (or symbols) are assigned to the same or corresponding components, and redundant descriptions thereof are omitted as appropriate for clarifying the explanation.

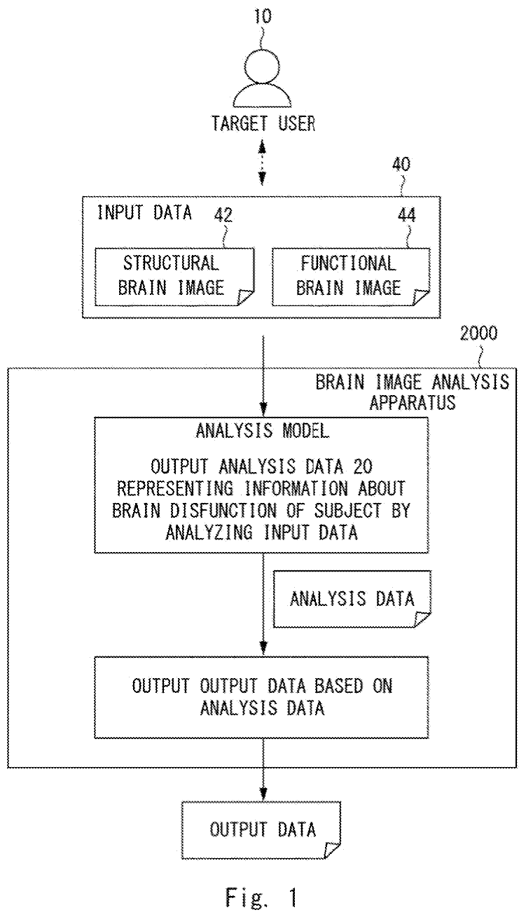

FIG. 1 shows an example of an overview of operations performed by a brain image analysis apparatus 2000 according to a first example embodiment. Note that FIG. 1 is a diagram for facilitating understanding of the overview of the brain image analysis apparatus 2000, and operations performed by the brain image analysis apparatus 2000 are not limited to those shown in FIG. 1.

The brain image analysis apparatus 2000 analyzes two or more types of images each of which includes the brain of a subject 10 and outputs output data 30 representing information about current or future brain dysfunction for the subject 10. For example, the output data 30 represents (but is not limited to) information shown below:
Whether or not the subject 10 has any brain dysfunction;
Whether or not the brain function of the subject 10 will worsen; or
Type of brain dysfunction that the subject 10 currently has or may have in the future.

Note that the subject 10 may be a person regarding which it is already known that they currently have brain dysfunction, or a person regarding which it is unknown as to whether or not they currently have brain dysfunction. Further, the subject 10 is not limited to people, but may be a non-human animal such as a dog.

A type of brain dysfunction that the brain image analysis apparatus 2000 handles is arbitrarily determined. For example, the brain dysfunction is mild dementia or Alzheimer's disease.

Images used by the brain image analysis apparatus 2000 include a structural brain image 42 and a functional brain image 44. The structural brain image 42 is an image showing the structure of the brain. The structural brain image 42 is, for example, a brain MRI (Magnetic Resonance Imaging) image or a brain CT (Computed Tomography) image. Meanwhile, the functional brain image 44 is an image showing the functional state of the brain. The functional brain image 44 is, for example, a brain PET (Positron Emission Tomography) image, a brain SPECT (Single Photon Emission Computed Tomography) image, or a diffusion tensor image.

The brain image analysis apparatus 2000 includes an analysis model 2020. The analysis model 2020 has been trained in advance so as to output analysis data 20 about the subject 10 in response to an input of input data 40 about the subject 10. The input data 40 includes a structural brain image 42 and a functional brain image 44 obtained by imaging the brain of the subject 10. The analysis data 20 represents information about current or future brain dysfunction of the subject 10. The analysis model 2020 is implemented by, for example, an analysis model generated by using heterogeneous mixture learning, a neural network, or an SVM (Support Vector Machine).

The brain image analysis apparatus 2000 acquires the input data 40 about the subject 10 including the structural brain image 42 and the functional brain image 44, and inputs the acquired input data 40 into the analysis model 2020. Then, the brain image analysis apparatus 2000 outputs output data 30 based on the analysis data 20 output from the analysis model 2020. The output data 30 may be the same as the analysis data 20, or may be data obtained by performing some processing on the analysis data 20. In the latter case, the output data 30 is, for example, a graph generated by using the analysis data 20.

### Example of Advantageous Effect

According to the brain image analysis apparatus 2000 in accordance with this example embodiment, by inputting the input data 40 of the subject 10 into the analysis model 2020, an analysis for the brain dysfunction of the subject 10 is made (e.g., whether the subject 10 has any brain dysfunction is determined). Here, the input data 40 includes the structural brain image 42 and the functional brain image 44, and the analysis model 2020 performs the analysis for the brain dysfunction of the subject 10 by using at least these two types of images. Therefore, according to the brain image analysis apparatus 2000 in accordance with this example embodiment, it is possible to make an analysis for the brain dysfunction of the subject 10 with high accuracy as compared with the case where an analysis is made by using only one type of brain image.

Further, since both of the structural brain image 42 and the functional brain image 44 are non-invasive data, the burden that is imposed on the subject 10 in order to acquire these data is small as compared with the burden imposed in order to acquire invasive data. Therefore, by making the analysis using the structural brain image 42 and the functional brain image 44, the analysis for the brain dysfunction of the subject 10 can be carried out with high accuracy while reducing the burden on the subject 10. However, although the analysis performed by the brain image analysis apparatus 2000 is required to at least use the structural brain image 42 and the functional brain image 44, it is not required not to use invasive data.

The brain image analysis apparatus 2000 according to this example embodiment will be described hereinafter in a more detailed manner.

### Example of Functional Configuration

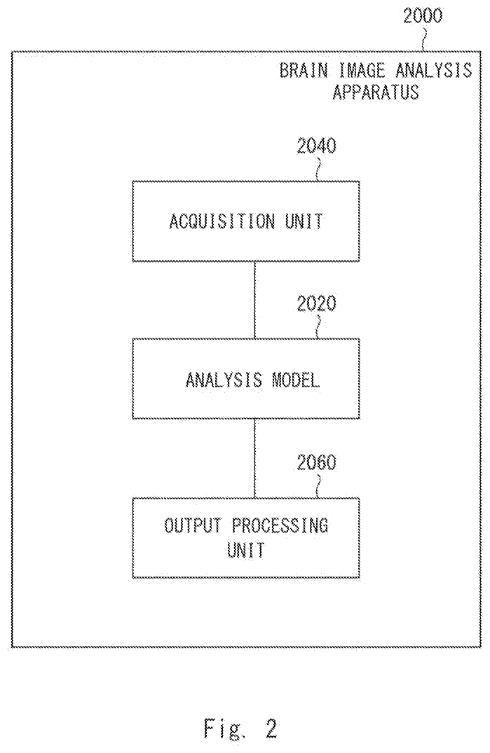

FIG. 2 is a block diagram showing an example of a functional configuration of the brain image analysis apparatus 2000 according to the first example embodiment. The brain image analysis apparatus 2000 includes an analysis model 2020, an acquisition unit 2040, and an output processing unit 2060. The analysis model 2020 outputs analysis data 20 of the subject 10 in response to an input of input data 40 of a subject 10. The acquisition unit 2040 acquires the input data 40 of the subject 10. The output processing unit 2060 acquires the analysis data 20 from the analysis model 2020 by inputting the input data 40 of the subject 10 into the analysis model 2020. Then, the output processing unit 2060 outputs the output data 30 based on the analysis data 20.

### Example of Hardware Configuration

Each functional component of the brain image analysis apparatus 2000 may be implemented by hardware (e.g., a hard-wired electronic circuit) that realizes the functional component, or by a combination of hardware and software (e.g., a combination of an electronic circuit and a program for controlling the electronic circuit). An example case where each functional component of the brain image analysis apparatus 2000 is implemented by a combination of hardware and software will be further described hereinafter.

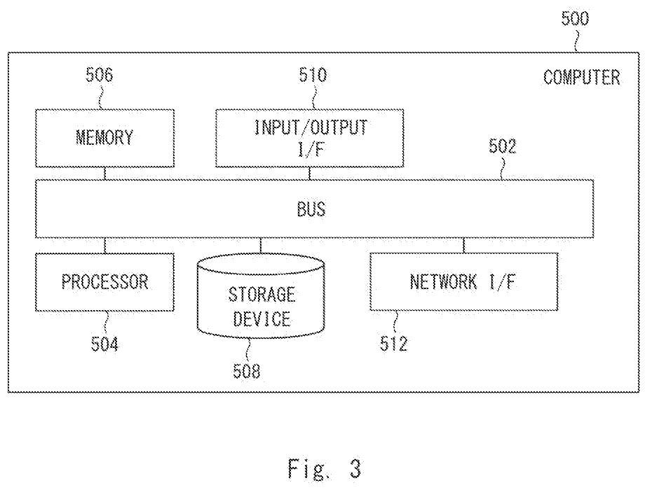

FIG. 3 is a block diagram showing an example of a hardware configuration of a computer 500 that implements the brain image analysis apparatus 2000. The computer 500 is any type of computer. For example, the computer 500 is a stationary computer such as a PC (Personal Computer) or a server machine. Alternatively, the computer 500 is, for example, a portable computer such as a smartphone or a tablet terminal. The computer 500 may be a dedicated computer designed to implement the brain image analysis apparatus 2000, or may be a general-purpose computer.

For example, each function of the brain image analysis apparatus 2000 is implemented by the computer 500 by installing a certain application program(s) in the computer 500. The aforementioned application program is composed of a program(s) for implementing the functional components of the brain image analysis apparatus 2000.

The computer 500 includes a bus 502, a processor 504, a memory 506, a storage device 508, an input/output interface 510, and a network interface 512. The bus 502 is a data transmission path through which the processor 504, the memory 506, the storage device 508, the input/output interface 510, and the network interface 512 transmit and receive data to and from each other. However, the method for connecting the processor 504 and the like to each other is not limited to connections through buses.

The processor 504 is one of various processors such as a CPU (Central Processing Unit), a GPU (Graphics Processing Unit), or an FPGA (Field-Programmable Gate Array). The memory 506 is a main storage device implemented by using a RAM (Random Access Memory) or the like. The storage device 508 is an auxiliary storage device implemented by using a hard disk drive, an SSD (Solid State Drive), a memory card, or a ROM (Read Only Memory).

The input/output interface 510 is an interface for connecting the computer 500 with an input/output device(s). For example, an input device such as a keyboard and an output device such as a display device are connected to the input/output interface 510.

The network interface 512 is an interface for connecting the computer 500 to a network. The network may be a LAN (Local Area Network) or a WAN (Wide Area Network).

The storage device 508 stores a program(s) for implementing each functional component of the brain image analysis apparatus 2000 (a program(s) for implementing the above-described application program). The processor 504 implements each functional component of the brain image analysis apparatus 2000 by loading the program(s) into the memory 506 and executing the loaded program(s).

The brain image analysis apparatus 2000 may be implemented by one computer 500, or may be implemented by a plurality of computers 500. In the latter case, the configurations of the computers 500 do not necessarily have to be identical to each other, i.e., may be different from each other.

### Flow of Processes

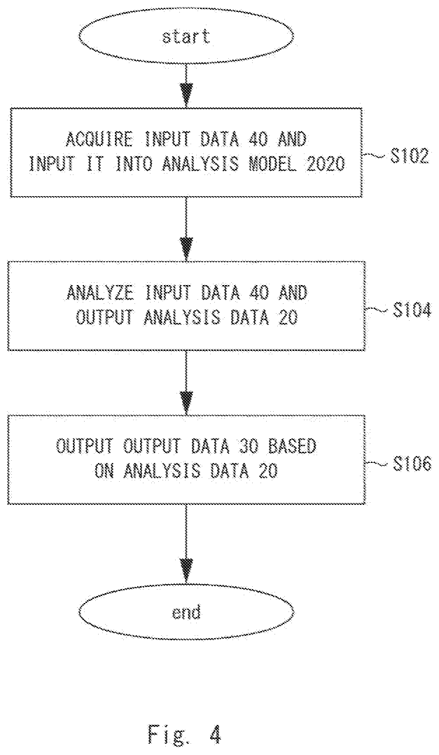

FIG. 4 is a flowchart showing an example of a flow of processes performed by the brain image analysis apparatus 2000 according to the first example embodiment. The acquisition unit 2040 acquires input data 40 and inputs the acquired input data 40 into the analysis model 2020 (S102). The analysis model 2020 processes the received input data 40 and outputs analysis data 20 (S104). The output processing unit 2060 outputs output data 30 based on the analysis data 20 (S106).

### Analysis by Analysis Model 2020

The analysis model 2020 performs an analysis for the brain dysfunction of the subject 10 by using the received input data 40, and outputs analysis data 20 representing a result of the analysis. Note that the analysis model 2020 has been trained in advance so as to output analysis data 20 in response to the input of the input data 40. A specific method for implementing the training of the analysis model 2020 will be described later.

The analysis performed by the analysis model 2020 is any of various analyses. An example of the analysis performed by the analysis model 2020 will be shown hereinafter in a concrete manner.

### Prediction about Current Brain Dysfunction

For example, the analysis model 2020 performs a prediction about the current brain dysfunction of the subject 10. For example, the analysis model 2020 calculates, by analyzing the input data 40, a probability that the subject 10 currently has brain dysfunction. In this case, for example, the analysis data 20 is a scalar value representing the probability that the subject 10 currently has brain dysfunction. In another example, the analysis model 2020 may determine whether or not the subject 10 currently has brain dysfunction. In this case, for example, the analysis data 20 is a flag indicating whether or not the subject 10 currently has brain dysfunction.

In another example, the analysis model 2020 predicts the type of brain dysfunction that the subject 10 currently has. In this case, for example, the analysis data 20 is vector data having the same number of elements as the number of types of brain dysfunction each of which the analysis model 2020 can determine whether the subject 10 has. Suppose that the analysis model 2020 can determine whether or not the subject 10 has mild dementia and whether or not the subject 10 has Alzheimer's disease. In this case, for example, the analysis data 20 is a vector data v=(p1, p2) that is composed of a probability p1 of having mild dementia and a probability p2 of having Alzheimer's disease. For example, when the analysis model 2020 outputs the analysis data 20 of (0.7, 0.1), this analysis data 20 indicates that “the probability that the subject 10 has mild dementia is 0.7 and the probability that the subject 10 has Alzheimer's disease is 0.1.”

### Prediction about Future Brain Dysfunction

For example, the analysis model 2020 performs a prediction about brain dysfunction of the subject 10 in the future. For example, the analysis model 2020 calculates a probability that the brain function of the subject 10 will worsen within a predetermined period. In this case, the analysis data 20 is a scalar value representing the probability that the brain function of the subject 10 will worsen within the predetermined period. In another example, the analysis model 2020 may determine whether or not the brain function of the subject 10 will worsen within the predetermined period. In this case, the analysis data 20 is a flag indicating whether or not the brain function of the subject 10 will worsen within the predetermined period.

It is noted that the fact that “the state of the brain function will worsen within a predetermined period” means that when the brain function of the subject 10 at the time when the structural brain image 42 and the functional brain image 44 are generated by imaging the brain of the subject 10 is compared with the brain function of the subject 10 at the time after the predetermined period has elapsed from the generation of the image, the brain function in the latter case has worsened from that in the former case. Examples of more specific cases include 1) a case where the state of the brain function changes from a state having no brain dysfunction to a state having a mild cognitive disorder, 2) a case where the state of the brain function changes from a state having no brain dysfunction to a state having an Alzheimer's disease state, and 3) a case where the state of the brain function changes from a state having mild cognitive disorder to a state having an Alzheimer's disease.

The analysis model 2020 may handle only a specific type of worsening (e.g., one of the above-described cases 1) to 3)), or may handle any type of worsening (e.g., considered as worsening when the case falls within any of the above-described cases 1) to 3)). Suppose that the analysis model 2020 handles the worsening of the case 3). In this case, input data 40 for a subject 10 having mild cognitive disorder is input into the analysis model 2020. Then, the analysis model 2020 outputs analysis data 20 representing a probability that the state of the subject 10 will change to the state having an Alzheimer's disease within a predetermined period (i.e., a probability that the state of the subject 10 will change from mild cognitive disorder to Alzheimer's disease within a predetermined period) or indicating whether or not the state of the subject 10 will change to the state having an Alzheimer's disease within a predetermined period.

In another example, the analysis model 2020 predicts a time until the brain function of the subject 10 worsens. In this case, for example, the analysis data 20 outputs a scalar value representing the time until the brain function of the subject 10 worsens. In another example, the analysis model 2020 may predict a probability that the time until the brain dysfunction of the subject 10 worsens falls within one of a plurality of alternatives (e.g., within one year, one to three years, three to five years, and five years or longer). In this case, for example, the analysis data 20 is a vector data having the same number of elements as the number of these alternatives.

Suppose that there are four alternatives: within one year; one to three years; three to five years; and five years or longer. In this case, for example, the analysis data 20 is a vector data v=(p1, p2, p3, p4) that enumerates four probabilities: a probability p1 that the brain function will worsen within one year; a probability p2 that the brain function will worsen over a period of one to three years; a probability p3 that the brain function will worsen over a period of three to five years; and a probability p4 that the brain function will not worsen within five years.

It is noted that, similarly to the case for predicting the probability of the worsening of the brain function, the analysis model 2020 in this case may handle only a specific type of worsening (e.g., one of the above-described cases 1) to 3)), or may handle any type of worsening.

In another example, the analysis model 2020 shows a type of brain dysfunction that the subject 10 is expected to have within a predetermined period in the future. In this case, for example, the analysis data 20 is a vector data having the same number of elements as the number of types of brain dysfunction each of which the analysis model 2020 can predict. Suppose that the analysis model 2020 can predict whether or not the subject 10 will have mild dementia within a predetermined period and whether or not the subject 10 will have Alzheimer's disease within a predetermined period. In this case, for example, the analysis data 20 is a vector v=(p1, p2) composed of a probability p1 that the subject 10 will have mild dementia within the predetermined period and a probability p2 that the subject 10 will have Alzheimer's disease within the predetermined period.

### Feature Value Extracted from Image

The analysis model 2020 extracts feature values from each of the structural brain image 42 and the functional brain image 44 included in the input data 40, and generates analysis data 20 by using the extracted feature values. The feature values extracted from the structural brain image 42 and those extracted from the functional brain image 44 are any of various feature values.

For example, the feature values extracted from the structural brain image 42 are feature values related to the size or shape of the whole or a part of the brain. Examples of more specific feature values include brain volume data such as volume data of the hippocampus, entorhinal cortex, forebrain, ventricle, or white-matter hyperintensity.

The feature values extracted from the functional brain image 44 vary depending on the type of the functional brain image 44. Suppose that the functional brain image 44 is a brain PET image in which a specific protein accumulated in the brain can be observed. In this case, the feature value extracted from the functional brain image 44 is a feature value related to the amount of a protein that can be observed. More specifically, examples of feature values include an average value of SUV (Standardized Uptake Value) ratios that is obtained by dividing the SUVs of four regions including the frontal cortex, anterior cingulate cortex, precuneus, and parietal lobe by the SUV of the cerebellum. It is noted that an example of the functional brain image 44 in which a specific protein can be observed as described above is a brain PET image that is obtained by using a chemical agent having a 18F-AV45 label and in which amyloid accumulated in the brain can be observed.

Further, suppose that the functional brain image 44 is a brain PET image in which carbohydrate metabolism in the brain can be observed. In this case, examples of feature values obtained from the functional brain image 44 include an average value of SUV ratios obtained by dividing the SUVs of three regions including the temporal cortex, anterior cingulate cortex, and posterior cingulate cortex by the SUV of the cerebellum. Such a functional brain image 44 can be obtained, for example, by using a chemical agent having a 18F-FDG label.

It is noted that although examples of feature values extracted from the structural brain image 42 and the functional brain image 44 have been described, some of the models which can be applied to the analysis model 2020 do not require an explicit specification of feature values to be extracted from the structural brain image 42 and the functional brain image 44, respectively. Examples of such models include a neural network.

### Case of Using Plurality of Structural Brain Images 42 or Functional Brain Images 44

The input data 40 may include a plurality of structural brain images 42. For example, it is possible to obtain a plurality of MRI images by imaging the brain of the subject 10 from each one of different angles, and input these MRI images into the analysis model 2020.

Further, the input data 40 may include a plurality of functional brain images 44. For example, as described above, it is possible to observe a plurality of different functions of the brain by using a plurality of chemical agents. In this case, it is possible to input functional brain images 44, each of which represents a respective one of different brain functions, into the analysis model 2020. For example, a functional brain image 44 in which a specific protein accumulated in the brain can be observed and a functional brain image 44 in which carbohydrate metabolism in the brain can be observed are input into the analysis model 2020.

It is noted that, as will be described later, when a plurality of structural brain images 42 and a plurality of functional brain images 44 are input into the analysis model 2020 as described above, a plurality of structural brain images 42 and a plurality of functional brain images 44 are also used in a similar manner at the time of the training.

### Case of Using Information Obtained from Something other than Image

The input data 40 may further include data obtained from something other the structural brain image 42 or the functional brain image 44. Examples of such data include attribute data, medical history data, health condition data, genetic data, and biomarker data of the subject 10.

Examples of the attribute data of the subject 10 include data representing the age, gender, and the history of education. Examples of the medical history data of the subject 10 include data indicating diseases from which the subject 10 suffered in the past and data indicating dates at which the subject 10 had the diseases. Examples of the health condition data of the subject 10 include data obtained in a medical checkup that the subject 10 underwent and data obtained in a medical examination and the like that were conducted when the subject 10 visited the hospital. More specifically, the examples include results of a blood test, an electrocardiogram test, and a urinalysis, or results of measurements of the height, weight, and blood pressure. As the genomic data of the subject 10, various data related to the genes of the subject 10 such as the base sequence of the genome of the subject 10 can be handled. As the biomarker data of the subject 10, data of various biomarkers obtained for the subject 10 can be handled. Examples of biomarkers include a cerebrospinal fluid biomarker and an inflammatory biomarker (such as progranulin and C-reactive protein).

Note that as will be described later, when various data other than brain images are also input into the analysis model 2020 as describe above, these data are also used in a similar manner at the time of the learning.

### Example in Heterogeneous Mixture Learning

An example of the configuration of the analysis model 2020 in a case where heterogeneous mixture learning is used will be shown hereinafter. Note that, in this example, it is assumed that the input data 40 includes only the structural brain image 42 and the functional brain image 44.

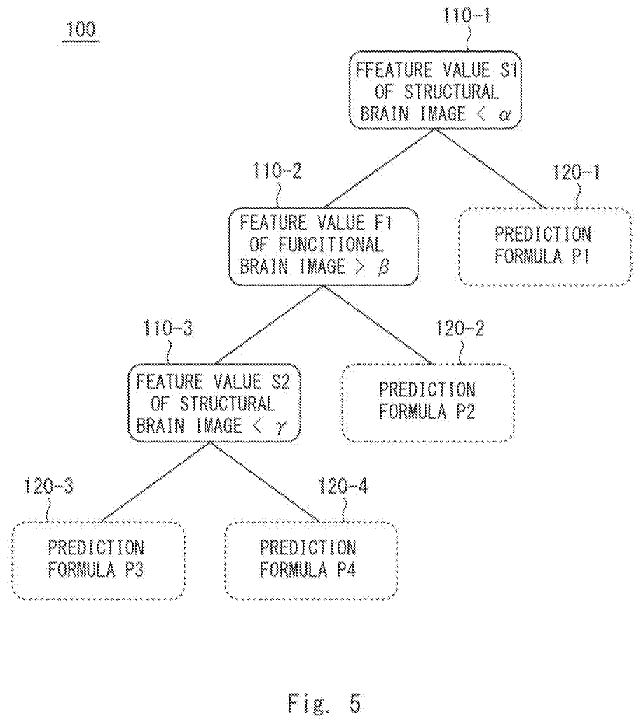

FIG. 5 shows an example of the analysis model 2020 generated by using heterogeneous mixture learning. In this example, the analysis model 2020 has a decision tree 100 having a depth of three. The decision tree 100 has branch nodes 110 and leaf nodes 120. Each of the branch nodes 110 represents a conditional branch based on a respective one of feature values obtained from the structural brain image 42, the functional brain image 44, or both thereof. Each of the leaf nodes 120 represents a prediction formula that is applied to the input data 40 that has reached that leaf node 120 through conditional branching by the branch node 110. Each prediction formula is an arbitrary model in which a respective one of feature values obtained from the structural brain image 42, the functional brain image 44, or both thereof is used as an explanatory variable: e.g., a linear regression model, a linear discriminant model, or a binomial logistic regression model, or the like. The prediction formula in each leaf node 120 is generated by using respective training data having a feature value that reaches that leaf node 120 through conditional branching by the decision tree 100.

When input data 40 is input into the analysis model 2020, the analysis model 2020 first determines a leaf node 120 to which the input data 40 reaches by performing conditional branching by the decision tree 100. Note that when the analysis model 2020 traces the decision tree 100, it extracts a feature value necessary for conditional branching from the input data 40. Then, the analysis model 2020 computes an analysis result by applying the feature value obtained from the input data 40 to the prediction formula corresponding to the determined leaf node 120.

### Acquisition of Input Data 40: S102

The acquisition unit 2040 acquires input data 40 for the subject 10 (S102). The method for obtaining the input data 40 is arbitrarily determined. For example, the input data 40 is stored in advance in a storage device in association with identification information of the subject 10. The acquisition unit 2040 acquires identification information of the subject 10 and acquires input data 40 associated with the acquired identification information.

Note that a plurality of data (the structural brain image 42, the functional brain image 44, and the like) constituting the input data 40 may be stored in storage devices different from each other. In this case, the acquisition unit 2040 acquires each of the plurality of data constituting the input data 40 by accessing the respective storage device.

It is noted that the method for acquiring the identification information of the subject 10 is also arbitrarily determined. For example, the identification information of the subject 10 is input by a user of the brain image analysis apparatus 2000. Alternatively, for example, the identification information of the subject 10 is read from a storage medium in which the identification information of the subject 10 is stored (such as a magnetic card or an IC card used as a patient's registration card) by using a reader.

### Generation of Output Information: S106

The output processing unit 2060 generates output data 30 by using the analysis data 20 obtained by inputting the input data 40 into the analysis model 2020 (S106). For example, the output data 30 is text data and/or image data (including screen data) generated based on the contents of the analysis data 20.

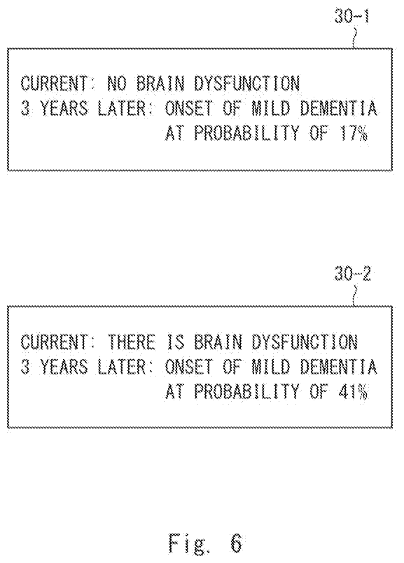

FIG. 6 shows an example of the output data 30. Output data 30-1 and 30-2 are both screen data output to a display device. The output data 30-1 shows that 1) the subject 10 currently has no brain dysfunction, and 2) there is a 17% probability of developing mild cognitive disorder within three years. Meanwhile, the output data 30-2 shows that 1) the subject 10 currently has mild cognitive disorder, and 2) there is a 41% probability of developing Alzheimer's disease within three years.

Note that, in the example shown in FIG. 6, a plurality of types of analysis models 2020 are used. Specifically, three models including 1) a first analysis model 2020 that predicts the type of brain disease the subject 10 currently has, 2) a second analysis model 2020 that predicts a probability of a transition from a state in which there is no brain dysfunction to a mild cognitive disorder state within three years, and 3) a third analysis model 2020 that predicts a probability of a transition from a mild cognitive disorder state to an Alzheimer's disease state within three years.

The second and third analysis models 2020 are selectively used based on the analysis data 20 output from the first analysis model 2020. That is, when it is determined that the subject 10 currently has no brain dysfunction by the analysis data 20, the second analysis model 2020 is used. On the other hand, when it is determined that the subject 10 currently has mild cognitive disorder by the analysis data 20, the third analysis model 2020 is used.

The output data 30 may be data representing the contents of the analysis data 20 as they are, or may be data obtained by performing some processing on the analysis data 20. For example, when the analysis data 20 is data representing a probability (e.g., data representing a probability that subject 10 has brain dysfunction), the output processing unit 2060 may make a determination by comparing the probability represented by the analysis data 20 with a threshold, and generate output data 30 indicating a result of the determination. Suppose that the analysis data 20 represents a probability that the subject 10 has brain dysfunction. In this case, when the probability represented by the analysis data 20 is equal to or higher than a threshold, the output processing unit 2060 generates output data 30 representing a determination result indicating that “the subject 10 has brain dysfunction”. On the other hand, when the probability is lower than the threshold, the output processing unit 2060 generates output data 30 representing a determination result indicating that the subject 10 has no brain dysfunction.

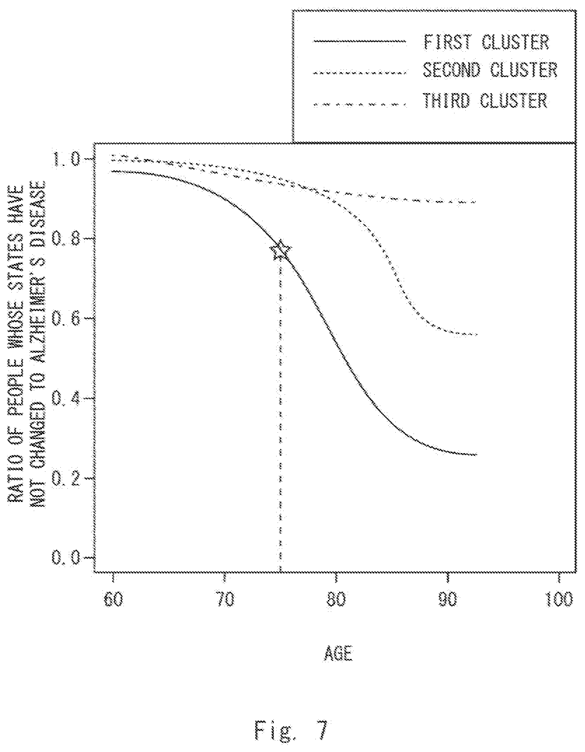

Further, for example, the output processing unit 2060 may generate a graph by using existing cases (training data used for the training of the analysis model 2020), and output, as the output data 30, a result of a comparison between a result of an analysis made for the subject 10 and the existing cases. FIG. 7 shows an example of a comparison between a result of an analysis made for the subject 10 and existing cases.

In this example, the analysis model 2020 is a model generated by using heterogeneous mixture learning, and its decision tree has three leaf nodes (i.e., three prediction formulas). In other words, in the analysis model 2020, the subject 10 is classified into one of the three clusters according to the decision tree. Then, a prediction for the subject 10 is made by using a prediction formula associated with the cluster into which the subject 10 has been classified.

The graph shown in FIG. 7 is obtained by classifying training data into clusters based on the decision tree included in the analysis model 2020, and generating a Kaplan-Meier curve for each of the clusters. The X-axis indicates ages, and the Y-axis indicates percentages of people whose states have changed from mild dementia to Alzheimer's disease. The solid curve is a Kaplan-Meier curve generated from cases classified in the first cluster. The dotted curve is a Kaplan-Meier curve generated from cases classified in the second cluster. The dashed curve is a Kaplan-Meier curve generated from cases classified in the third cluster.

In this example, the input data 40 for the subject 10 is input into the analysis model 2020, and as a result, the subject 10 is classified into the first cluster. Further, it is predicted that a time over which the state of the subject 10 will change from mild dementia to Alzheimer's disease is five years. Suppose that the subject 10 is currently 70 years old. Therefore, the age at which the state of the subject 10 is expected to change from mild cognitive disorder to Alzheimer's disease is 75 years.

From this result, the analysis result for the subject 10 corresponds to a point “Age=75 years” in the Kaplan-Meier curve for the first cluster. Therefore, in FIG. 7, a plot having a star-like shape is displayed at this point.

By displaying the analysis result for the subject 10 and the comparison result with existing cases as described above, the user can visually and easily recognize information such as 1) the state of the subject 10 is expected to change from mild cognitive disorder to Alzheimer's disease at the age of 75 years, 2) the subject 10 is classified into the first cluster, and 3) the relationship between the age of people and the ratio of the transition to Alzheimer's disease regarding the people who are classified into the first cluster like the subject 10.

The output destination of the output data 30 is arbitrarily determined. For example, the output data 30 is displayed in a display device that can be accessed from the output processing unit 2060. In another example, the output data 30 is stored in a storage device that can be accessed from the output processing unit 2060. In another example, the output data 30 is transmitted from the output processing unit 2060 to other apparatuses. For example, it is possible that when input data 40 is transmitted from a user terminal to the brain image analysis apparatus 2000, output data 30 obtained by analyzing this input data 40 is transmitted from the output processing unit 2060 to the user terminal.

### Training of Analysis Model 2020

The analysis model 2020 is trained in advance before it is used by the brain image analysis apparatus 2000. Hereafter, an apparatus by which the analysis model 2020 is trained is called a training apparatus. The training apparatus may be provided integrally with the brain image analysis apparatus 2000, or may be provided separately from the brain image analysis apparatus 2000. In the former case, the training apparatus is implemented by the computer that implements the brain image analysis apparatus 2000. On the other hand, in the latter case, the training apparatus is implemented by a computer different from the computer that implements the brain image analysis apparatus 2000. It is noted that, in the latter case, the computer that implements the training apparatus can be any computer and has, for example, a hardware configuration shown in FIG. 3.

The training apparatus acquires a plurality of training data, and trains the analysis model 2020 by using the plurality of training data. The training data includes a combination of “input data 40, a ground-truth analysis data 20 (i.e., analysis data 20 that should be output from the analysis model 2020 when the input data 40 is input into the analysis model 2020)”. The training data can be generated by using case data. The case data is data obtained from examinations and the like performed on certain persons or animals (hereinafter referred to as case patients). Hereinafter, regarding the various types of the analysis model 2020 mentioned above, the training data used for the training will be described. It is noted that various existing technologies can be used for the technology for training a model by using training data.

Suppose that the analysis model 2020 has already been trained so as to predict a probability that the subject 10 currently has brain dysfunction. In this case, training data can be generated from case data of case patients who were diagnosed as having brain dysfunction, case data of case patients who were diagnosed as having no brain dysfunction, or each of both of these case data. From the case patients diagnosed as having brain dysfunction, training data (positive example training data) that includes a combination of “input data 40 obtained for these case patients, a probability of having brain dysfunction=1” is obtained. On the other hand, from the case patients diagnosed as having no brain dysfunction, leaning data (negative example training data) that includes a combination of “input data 40 obtained for these case patients, a probability of having brain dysfunction=0” is obtained. The analysis model 2020 is trained by using these training data. It is noted that training of the analysis model 2020 that predicts whether or not the subject 10 currently has brain dysfunction can be carried out by using similar training data.

In another example, suppose that the analysis model 2020 has been trained so as to predict the type of brain dysfunction that subject 10 currently has. In this case, for example, the analysis model 2020 is trained so as to output, for each type of brain dysfunction, vector data indicating a probability that the subject 10 has that type of brain dysfunction. In this case, the training data generated from the case data indicates, as ground-truth analysis data 20, “vector data that indicates a probability of 1 for types of brain dysfunction that the case patient has, and a probability of 0 for the other types of brain dysfunction”.

Suppose that the analysis model 2020 has been trained so as to output a vector v=(p1, p2), where p1 represents a probability that the subject 10 has mild cognitive disorder, and p2 represents a probability that the subject 10 has Alzheimer's disease. In this case, from case data of a case patient who has mild cognitive disorder but has no Alzheimer's disease, training data of “input data 40 obtained for this case patient, v=(1, 0)” can be generated.

In another example, suppose that the analysis model 2020 has been trained so as to predict a probability that the brain function of the subject 10 will worsen within a predetermined period. In this case, training data can be generated from case data including results of examinations and the like performed at the time when data used as input data 40 was generated (when the structural brain image 42 and the functional brain image 44 were generated by imaging the brain) and those that were obtained at the time after a predetermined period has elapsed from the generation of the data. For example, from case data indicating that the brain function worsened within a predetermined period, leaning data (positive example training data) that includes a combination of “input data 40 obtained for this case patient, a probability that the brain function will worsen=1” is obtained. On the other hand, from case data indicating the brain function did not worsen within the predetermined period, leaning data (negative example training data) that includes a combination of “input data 40 obtained for this case patient, a probability that the brain function will worsen=0” is obtained. The analysis model 2020 is trained by using these training data. It is noted that the training of the analysis model 2020 that predicts whether or not the subject 10 will worsen within a predetermined period can be carried out by using similar training data.

Note that, as described above, it is possible to handle only specific worsening as the worsening of the brain function. Suppose that the worsening of “changing from a state of having a mild cognitive disorder to a state of having an Alzheimer's disease” is handled. In this case, from case data of a case patient indicating that the subject 10 was diagnosed as having mild cognitive disorder at the examination at the time when the data to be used as input data 40 was generated, and then the subject 10 was diagnosed as having Alzheimer's disease at the examination that was performed a predetermined period after the generation of the data, training data of “input data 40 obtained for this case patient, a probability that the brain function will worsen=1” is obtained. On the other hand, from case data of a case patient indicating that the subject 10 was diagnosed as having mild cognitive disorder at the examination at the time when the data to be used as input data 40 was generated, and then the subject 10 was diagnosed as having no Alzheimer's disease at the examination that was performed the predetermined period after the generation of the data, training data of “input data 40 obtained for this case patient, a probability that the brain function will worsen=0” is obtained.

In another example, suppose that, for example, the analysis model 2020 predicts a time until the brain function of the subject 10 worsens. In this case, it is assumed that case data of a certain case patient indicates that the brain function has worsened based on results of examinations conducted at the time when data to be used as input data 40 was generated and those after a time t had elapsed from the generation of the data. In this case, training data of “input data 40 obtained for this case patient, the time until the brain function worsens=t” is obtained from the above-described case data. Then, the analysis model 2020 is trained by using this training data.

It is noted that, as described above, the time until the brain function of the subject 10 worsens may be expressed by using a plurality of alternatives, such as within one year and one to three years. In this case, training data can be generated by classifying the time t until the brain function worsens, which is represented by the case data, into one of these alternatives. Suppose that the analysis data 20 is vector data v=(p1, p2, p3, p4) comprising four probabilities: a probability p1 that the brain function will worsen within one year; a probability p2 that the brain function will worsen over a period of one to three years; a probability p3 that the brain function will worsen over a period of three to five years; and a probability p4 that the brain function will not worsen within five years. In this case, from case data of a case patient who was diagnosed that the brain function worsened over a period of seven months, training data of “input data 40 obtained for this case patient, v=(1, 0, 0, 0)” is obtained. Further, from case data of a case patient whose brain function had not worsened even after five years had elapsed, training data of “input data 40 obtained for this case patient, v=(0, 0, 0, 1)” is obtained.

In another example, the analysis model 2020 is trained so as to predict the type of brain dysfunction that the subject 10 is expected to have within a predetermined period in the future. In this case, for example, the analysis model 2020 is trained so as to output, for each type of brain dysfunction, vector data indicating a probability that the subject 10 will have that type of brain dysfunction within a predetermined period in the future. In this case, training data can be generated from case data including results of examinations conducted at the time when data to be used as input data 40 was generated and those at the time after a predetermined time had elapsed from the generation of the data. The training data indicates, as ground-truth analysis data 20, “vector data indicating a probability of 1 for a type(s) of brain dysfunction that the patient had at the time of the examination conducted after the predetermined period, and a probability of 0 for the other types of brain dysfunctions”.

Suppose that the analysis model 2020 has been trained so as to output a vector v=(p1, p2), where p1 represents a probability that the subject 10 has mild cognitive disorder, and p2 represents a probability that the subject 10 has Alzheimer's disease. In this case, from case data of a case patient who was diagnosed as having mild cognitive disorder but having no Alzheimer's disease in the examination conducted after the predetermined period, training data of “input data 40 obtained for this case patient, v=(1, 0)” can be generated.

### Output of Information obtained through Training

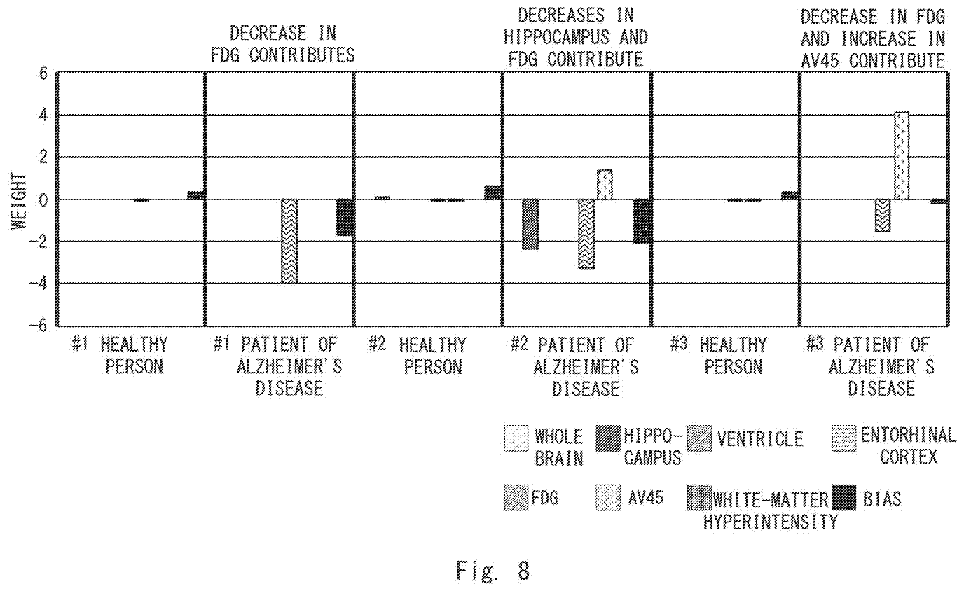

The output processing unit 2060 of the brain image analysis apparatus 2000 or the training apparatus may have a function of outputting information obtained through the training of the analysis model 2020. FIG. 8 is a first diagram showing an example of information obtained through training of the analysis model 2020. In this example, the analysis model 2020 has been trained through heterogeneous mixture learning. More specifically, in this analysis model 2020, training data are divided into three clusters, and for each of the three clusters, two prediction formulas are generated. The prediction formulas generated for each cluster includes a first prediction formula for calculating a score representing a probability that the subject is healthy person (hereinafter also referred to as a healthy-person score) and a second prediction formula for calculating a score representing a probability that the subject has Alzheimer's disease (hereinafter also referred to as an AD score).

The analysis model 2020 determines a cluster to which the subject 10 belongs by using a feature value(s) obtained from the input data 40, and applies the feature value(s) obtained from the input data 40 to each of the first and second prediction formulas associated with the specified cluster. In this way, the healthy-person score and the AD score for the subject 10 are obtained. The analysis model 2020 determines whether the subject 10 is a healthy person or a patient having Alzheimer's disease by comparing the healthy-person score with the AD score, and outputs analysis data 20 indicating the result of the determination.

In this example, the input data 40 includes three images: a brain MRI image; a brain PET image in which carbohydrate metabolism in the brain can be observed; and a brain PET image in which amyloid accumulated in the brain can be observed. In the graph shown in FIG. 8, FDG represents a feature value obtained from the brain PET image in which carbohydrate metabolism in the brain can be observed. Further, AV45 represents a feature value obtained from the brain PET image in which amyloid accumulated in the brain can be observed. Further, the whole brain, hippocampus, ventricle, entorhinal cortex, and white-matter hyperintensity are feature values representing the sizes of these regions, respectively, obtained from the brain MRI. Further, the prediction formula includes a bias term.

The graph shown in FIG. 8 shows, from left to right, weights for respective feature values (explanatory variables) for six prediction formulas including 1) a first prediction formula in the first cluster, 2) a second prediction formula in the first cluster, 3) a first prediction formula in the second cluster, 4) a second prediction formula in the second cluster, 5) a first prediction formula in the third cluster, and 6) a second prediction formula in the third cluster. By using this graph, it is possible to realize the magnitude of the contribution of feature values to each of the likelihood of being a healthy parson and that of being a person having Alzheimer's disease.

For example, for the first cluster, it can be understood that a decrease in FDG contributes to the likelihood of being a patient having Alzheimer's disease. Further, for the second cluster, it can be understood that decreases in hippocampus and FDG, and an increase in AV45 contribute to the likelihood of being a patient having Alzheimer's disease. Further, for the third cluster, it can be understood that a decrease in FDG, and an increase in AV45 contribute to the likelihood of being a patient having Alzheimer's disease. It is noted that, in FIG. 8, such explanations are displayed on the graph. However, such explanations do not necessarily have to be displayed.

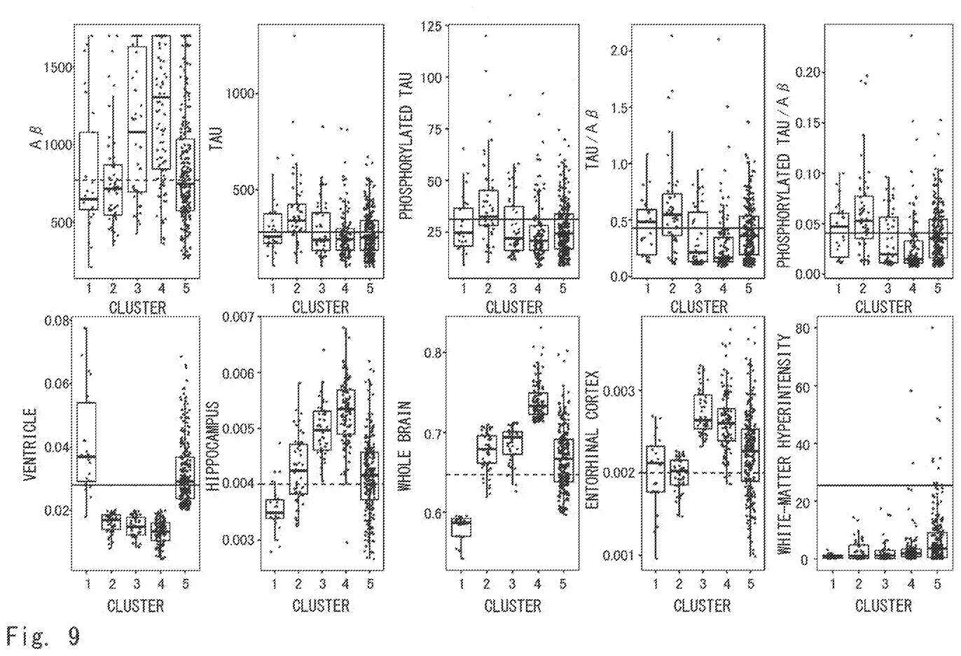

FIG. 9 is a second diagram showing an example of information obtained through training of the analysis model 2020. In this example, the analysis model 2020 is trained through heterogeneous mixture learning, so that the training data are divided into five clusters. Further, the graph shown in FIG. 9 shows, after the training data are classified into respective clusters, for each type of feature values, a distribution of feature values in each training data. For example, in the graph of Aβ, for each cluster, a distribution of Aβ values in the training data classified in that cluster is shown. It is noted that all training data were generated from case data of case patients having mild cognitive disorder.

Further, the solid and dotted thick horizontal lines represent cutoff values for distinguishing between healthy people and patients having Alzheimer's disease. The cutoff values are values at which the accuracy for distinguishing healthy people from AD patients (=(true positive+true negative)/total number) is maximized. In the case indicated by the solid line, it is indicated that when values are larger than the cutoff value, the subject is likely to have Alzheimer's disease (the distribution is closer to the distribution of only Alzheimer's patients than to that of only healthy people). On the other hand, in the case indicted by the dotted line, it is indicated that when the value is smaller than the cutoff value, the subject is likely to have Alzheimer's disease.

From these graphs, for example, it is possible to find out whether the case patients classified in the respective clusters are those who are close to healthy people (the worsening of the brain function is relatively small) or those who are close to Alzheimer's disease (the worsening of the brain function is relatively large). For example, in FIG. 9, the distribution for the first cluster shows that the AP and the tau ratio are abnormal and brain atrophy is also advanced. Therefore, it can be understood that many of the case patients classified in the first cluster have mild dementia close to Alzheimer's disease. Further, the distributions for the third and fourth clusters show that the AP and tau are normal and brain atrophy is not advanced. Therefore, it can be understood that many of the case patients classified in these clusters have mild dementia close to healthy people.

It is noted that, as described above, the output destination of various information obtained through the training is arbitrarily determined as in the case of the output destination of the output data 30.

Although the present invention is described above with reference to example embodiments, the present invention is not limited to the above-described example embodiments. Various modifications that can be understood by those skilled in the art can be made to the configuration and details of the present invention within the scope of the invention.

Note that, in the above-described examples, the program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g., magneto-optical disks), CD-ROM, CD-R, CD-R/W, and semiconductor memories (such as mask ROM, PROM (Programmable ROM), EPROM (Erasable PROM), flash ROM, RAM, etc.). Further, the program may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

The whole or part of the embodiments disclosed above can be described as, but not limited to, the following supplementary notes.

(Supplementary note 1)

A brain image analysis apparatus comprising:

an analysis model that has been trained to output analysis data representing information about brain dysfunction in response to an input of input data including a structural brain image showing a structure of a brain and a functional brain image showing a functional state of the brain;

an acquisition unit configured to acquire the input data for a subject; and

an output processing unit configured to acquire the analysis data about the subject from the analysis model by inputting the acquired input data into the analysis model, and to output output data representing information about brain dysfunction of the subject based on the acquired analysis data.

(Supplementary note 2)

The brain image analysis apparatus described in Supplementary note 1,

wherein the analysis model generates analysis data about at least one of a probability that the subject currently has brain dysfunction, a type of the brain dysfunction the subject currently has, a type of the brain dysfunction the subject will have within a predetermined period, a probability that a brain function of the subject will worsen within a predetermined period, or a time until the brain function of the subject worsens.

(Supplementary note 3)

The brain image analysis apparatus described in Supplementary note 2,

wherein the type of the brain dysfunction includes one or both of mild cognitive disorder and Alzheimer's disease.

(Supplementary note 4)

The brain image analysis apparatus described in Supplementary note 3,

wherein the subject has mild cognitive disorder, and

the analysis model generates analysis data about a probability that a state of the subject will change from the mild cognitive disorder to Alzheimer's disease within a predetermined period.

(Supplementary note 5)

The brain image analysis apparatus described in any one of Supplementary notes 1 to 4,

wherein the analysis model generates the analysis data by further using at least one of data about an attribute of the subject, data about a medical history of the subject, data about a health condition of the subject, genomic data of the subject, or biomarker data obtained from the subject.

(Supplementary note 6)

The brain image analysis apparatus described in any one of Supplementary notes 1 to 5,

wherein in training of the analysis model, a plurality of training data are classified into a plurality of clusters based on a feature value obtained from the training data, and a prediction formula is generated for each of the clusters, and

the analysis model determines, based on the feature value obtained from input data of the subject, the cluster to which this feature value belongs, and generates the analysis data by applying the feature value obtained from the input data of the subject to the prediction formula corresponding to the specified cluster.

(Supplementary note 7)

The brain image analysis apparatus described in Supplementary note 6,

wherein the analysis model is a model generated through training using heterogeneous mixture learning.

(Supplementary note 8)

The brain image analysis apparatus described in Supplementary note 6 or 7,

wherein the output processing unit performs:
generating, for each of the clusters, a graph of brain dysfunction of case patients of training data belonging to the cluster; and
generating the output data by using the analysis data obtained for the subject, the output data being obtained by superimposing data about the brain dysfunction of the subject onto the graph of the cluster to which the subject belongs.
   (Supplementary note 9)

The brain image analysis apparatus described in any one of Supplementary notes 6 to 8,

wherein the output processing unit outputs, for each of a plurality of types of feature values obtained from the training data, a graph showing, for each of the clusters, a distribution of the training data.

(Supplementary note 10)

The brain image analysis apparatus described in any one of Supplementary notes 6 to 9,

wherein the analysis model includes, for each of the clusters, a first prediction formula representing a probability that the subject is a healthy person and a second prediction formula representing a probability that the subject has brain dysfunction,

in both the first and second prediction formulas, a weight is assigned to each of a plurality of feature values obtained from the training data, and

the output processing unit outputs a graph showing the weights assigned to respective feature values for each of the first and second prediction formulas provided for each of the clusters.

(Supplementary note 11)

A control method performed by a computer,

wherein the computer comprises an analysis model that has been trained to output analysis data representing information about brain dysfunction in response to an input of input data including a structural brain image showing a structure of a brain and a functional brain image showing a functional state of the brain, and

the control method comprises:

an acquisition step for acquiring the input data for a subject; and

an output processing step for acquiring the analysis data about the subject from the analysis model by inputting the acquired input data into the analysis model, and outputting output data representing information about brain dysfunction of the subject based on the acquired analysis data.

(Supplementary note 12)

The control method described in Supplementary note 11,

wherein the analysis model generates analysis data about at least one of a probability that the subject currently has brain dysfunction, a type of the brain dysfunction the subject currently has, a type of the brain dysfunction the subject will have within a predetermined period, a probability that a brain function of the subject will worsen within a predetermined period, and a time until the brain function of the subject worsens.

(Supplementary note 13)

The control method described in Supplementary note 12,

wherein the type of the brain dysfunction includes one or both of mild cognitive disorder and Alzheimer's disease.

(Supplementary note 14)

The control method described in Supplementary note 13,

wherein the subject has mild cognitive disorder, and

the analysis model generates analysis data about a probability that a state of the subject will change from the mild cognitive disorder to Alzheimer's disease within a predetermined period.

(Supplementary note 15)

The control method described in any one of Supplementary notes 11 to 14,

wherein the analysis model generates the analysis data by further using at least one of data about an attribute of the subject, data about a medical history of the subject, data about a health condition of the subject, genomic data of the subject, or biomarker data obtained from the subject.

(Supplementary note 16)

The control method described in any one of Supplementary notes 11 to 15,

wherein in learning of the analysis model, a plurality of training data are classified into a plurality of clusters based on a feature value obtained from the training data, and a prediction formula is generated for each of the clusters, and

the analysis model determines, based on the feature value obtained from input data of the subject, the cluster to which this feature value belongs, and generates the analysis data by applying the feature value obtained from the input data of the subject to the prediction formula corresponding to the specified cluster.

(Supplementary note 17)

The control method described in Supplementary note 16,

wherein the analysis model is a model generated through training using heterogeneous mixture learning.

(Supplementary note 18)

The control method described in Supplementary note 16 or 17,

wherein in the output processing step,

generating, for each of the clusters, a graph of brain dysfunction of case patients of training data belonging to the cluster; and

generating the output data by using the analysis data obtained for the subject, the output data being obtained by superimposing data about the brain dysfunction of the subject onto the graph of the cluster to which the subject belongs.

(Supplementary note 19)

The control method described in any one of Supplementary notes 16 to 18,

wherein, in the output processing step, for each of a plurality of types of feature values obtained from the training data, a graph showing, for each of the clusters, a distribution of the training data is output.

(Supplementary note 20)

The control method described in any one of Supplementary notes 16 to 19,

wherein the analysis model includes, for each of the clusters, a first prediction formula representing a probability that the subject is a healthy person and a second prediction formula representing a probability that the subject has brain dysfunction,

in both the first and second prediction formulas, a weigh is assigned to each of a plurality of feature values obtained from the training data, and

in the output processing step, outputting a graph showing the weights assigned to respective feature values for each of the first and second prediction formulas provided for each of the clusters.

(Supplementary note 21)

A computer readable medium storing a program executed by a computer,

wherein the computer comprises an analysis model that has been trained to output analysis data representing information about brain dysfunction in response to an input of input data including a structural brain image showing a structure of a brain and a functional brain image showing a functional state of the brain, and

the program causes the computer to perform:

an acquisition step for acquiring the input data for a subject; and

an outputting step for acquiring the analysis data about the subject from the analysis model by inputting the acquired input data into the analysis model, and outputting output data representing information about brain dysfunction of the subject based on the acquired analysis data.

(Supplementary note 22)

The computer readable medium described in Supplementary note 21,

wherein the analysis model generates analysis data about at least one of a probability that the subject currently has brain dysfunction, a type of the brain dysfunction the subject currently has, a type of the brain dysfunction the subject will have within a predetermined period, a probability that a brain function of the subject will worsen within a predetermined period, or a time until the brain function of the subject worsens.

(Supplementary note 23)

The computer readable medium described in Supplementary note 22, wherein the type of the brain dysfunction includes one or both of mild cognitive disorder and Alzheimer's disease.

(Supplementary note 24)

The brain image analysis apparatus described in Supplementary note 23,

wherein the subject has mild cognitive disorder, and

the analysis model generates analysis data about a probability that a state of the subject will change from the mild cognitive disorder to Alzheimer's disease within a predetermined period.

(Supplementary note 25)

The computer readable medium described in any one of Supplementary notes 21 to 24,

wherein the analysis model generates the analysis data by further using at least one of data about an attribute of the subject, data about a medical history of the subject, data about a health condition of the subject, genomic data of the subject, or biomarker data obtained from the subject.

(Supplementary note 26)

The brain image analysis apparatus described in any one of Supplementary notes 21 to 25,

wherein in training of the analysis model, a plurality of training data are classified into a plurality of clusters based on a feature value obtained from the training data, and a prediction formula is generated for each of the clusters, and

the analysis model determines, based on the feature value obtained from input data of the subject, the cluster to which this feature value belongs, and generates the analysis data by applying the feature value obtained from the input data of the subject to the prediction formula corresponding to the specified cluster.

(Supplementary note 27)

The computer readable medium described in Supplementary note 26,

wherein the analysis model is a model generated through learning using heterogeneous mixture learning.

(Supplementary note 28)

The computer readable medium described in Supplementary note 26 or 27,

wherein in the outputting step,

generating, for each of the clusters, a graph of brain dysfunction of case patients of training data belonging to the cluster; and

generating the output data by using the analysis data obtained for the subject, the output data being obtained by superimposing data about the brain dysfunction of the subject onto the graph of the cluster to which the subject belongs.

(Supplementary note 29)

The computer readable medium described in any one of Supplementary notes 26 to 28,

wherein, in the output processing step, for each of a plurality of types of feature values obtained from the training data, a graph showing, for each of the clusters, a distribution of the training data is output.

(Supplementary note 30)

The brain image analysis apparatus described in any one of Supplementary notes 26 to 29,

wherein the analysis model includes, for each of the clusters, a first prediction formula representing a probability that the subject is a healthy person and a second prediction formula representing a probability that the subject has brain dysfunction,

in both the first and second prediction formulas, a weight is assigned to each of a plurality of feature values obtained from the training data, and

in the output processing step, outputting a graph showing the weights assigned to respective feature values for each of the first and second prediction formulas provided for each of the clusters.

### REFERENCE SIGNS LIST

10 SUBJECT
20 ANALYSIS DATA
30 OUTPUT DATA
40 INPUT DATA
42 STRUCTURAL BRAIN IMAGE
44 FUNCTIONAL BRAIN IMAGE
100 DECISION TREE
110 BRANCH NODE
120 LEAF NODE
500 COMPUTER
502 BUS
504 PROCESSOR
506 MEMORY
508 STORAGE DEVICE
510 INPUT/OUTPUT INTERFACE
512 NETWORK INTERFACE
2000 BRAIN IMAGE ANALYSIS APPARATUS
2020 ANALYSIS MODEL
2040 ACQUISITION UNIT
2060 OUTPUT PROCESSING UNIT

1. A brain image analysis apparatus comprising:
at least one memory storing instructions; and
at least one processor,
wherein the at least one memory further stores an analysis model that has been trained to output analysis data representing information about brain dysfunction in response to an input of input data including a structural brain image showing a structure of a brain and a functional brain image showing a functional state of the brain, and
the at least one processor is configured to execute:
acquire the input data for a subject;
acquire the analysis data about the subject from the analysis model by inputting the acquired input data into the analysis model; and
output output data representing information about brain dysfunction of the subject based on the acquired analysis data.

2. The brain image analysis apparatus according to claim 1,
wherein the analysis model generates analysis data about at least one of a probability that the subject currently has brain dysfunction, a type of the brain dysfunction the subject currently has, a type of the brain dysfunction the subject will have within a predetermined period, a probability that a brain function of the subject will worsen within a predetermined period, or a time until the brain function of the subject worsens.

3. The brain image analysis apparatus according to claim 2,
wherein the type of the brain dysfunction includes one or both of mild cognitive disorder and Alzheimer's disease.

4. The brain image analysis apparatus according to claim 3,
wherein when the subject has mild cognitive disorder,
the analysis model generates analysis data about a probability that a state of the subject will change from the mild cognitive disorder to Alzheimer's disease within a predetermined period.

5. The brain image analysis apparatus according to claim 1,
wherein the analysis model generates the analysis data by further using at least one of data about an attribute of the subject, data about a medical history of the subject, data about a health condition of the subject, genomic data of the subject, or biomarker data obtained from the subject.

6. The brain image analysis apparatus according to claim 1,
wherein in training of the analysis model, a plurality of training data are classified into a plurality of clusters based on a feature value obtained from the training data, and a prediction formula is generated for each of the clusters, and
the analysis model determines, based on the feature value obtained from input data of the subject, the cluster to which this feature value belongs, and generates the analysis data by applying the feature value obtained from the input data of the subject to the prediction formula corresponding to the specified cluster.

7. The brain image analysis apparatus according to claim 6,
wherein the analysis model is a model generated through training using heterogeneous mixture learning.

8. The brain image analysis apparatus according to claim 6,
wherein the output of the output data includes:
generating, for each of the clusters, a graph of brain dysfunction of case patients of the training data belonging to the cluster; and
generating the output data by using the analysis data obtained for the subject, the output data being obtained by superimposing data about the brain dysfunction of the subject onto the graph of the cluster to which the subject belongs.

9. The brain image analysis apparatus according to claim 6,
wherein the output of the output data includes outputting, for each of a plurality of types of feature values obtained from the training data, a graph showing, for each of the clusters, a distribution of the training data.

10. The brain image analysis apparatus according to claim 6,
wherein the analysis model includes, for each of the clusters, a first prediction formula representing a probability that the subject is a healthy person and a second prediction formula representing a probability that the subject has brain dysfunction,
in both the first and second prediction formulas, a weigh factor is assigned to each of a plurality of feature values obtained from the training data, and
the output of the output data includes: outputting a graph showing the weighs assigned to respective feature values for each of the first and second prediction formulas provided for each of the clusters.

11. A control method performed by a computer,
wherein the computer comprises an analysis model that has been trained to output analysis data representing information about brain dysfunction in response to an input of input data including a structural brain image showing a structure of a brain and a functional brain image showing a functional state of the brain, and
the control method comprises:
acquiring the input data for a subject;
acquiring the analysis data about the subject from the analysis model by inputting the acquired input data into the analysis model; and
outputting output data representing information about brain dysfunction of the subject based on the acquired analysis data.

12.-20. (canceled)

21. A computer readable medium storing a program executed by a computer,
wherein the program comprises an analysis model that has been trained to output analysis data representing information about brain dysfunction in response to an input of input data including a structural brain image showing a structure of a brain and a functional brain image showing a functional state of the brain, and
the program causes the computer to perform:
acquiring the input data for a subject;
acquiring the analysis data about the subject from the analysis model by inputting the acquired input data into the analysis model; and
outputting output data representing information about brain dysfunction of the subject based on the acquired analysis data.

22.-30. (canceled)
